(19) United States
(12) Patent Application Publication
(10) Pub. No.: US 2007/0065468 A1
(43) Pub. Date: Mar. 22, 2007

(54) Chimeric allergens for immunotherapy

(75) Inventors: Kaw Yan Chua, Kent Vale (SG); Lip Nyin Liew, Sandakan (MY); Lay Hong Lim, Jelapang (SG)

Correspondence Address: MORRISON & FOERSTER LLP, 425 MARKET STREET, SAN FRANCISCO, CA, 94105-2482, US

(73) Assignee: NATIONAL UNIVERSITY OF SINGAPORE, Kent Ridge Crescent (SG)

(21) Appl. No.: 11/512,953

(22) Filed: Aug. 29, 2006

Related U.S. Application Data
(60) Provisional application No. 60/712,271, filed on Aug. 29, 2005.

(51) Int. Cl. A61K 39/35; C07K 14/435

(52) U.S. Cl. 424/275.1; 530/350

(57) ABSTRACT

The present invention provides a chimeric polypeptide for reducing specific IgE binding to at least two house dust mite allergens in a subject, said chimeric polypeptide comprises an amino acid sequence comprising sequences of said at least two house dust mite allergens and wherein upon exposure to said allergens, said reduced binding causes reduced allergic reaction to said allergens in said subject.

### TECHNICAL FIELD

The present invention generally relates to immunology. The present invention also relates to the production and use of chimeric polypeptides for immunotherapy.

### BACKGROUND

The increase in the prevalence of allergic diseases in developed countries such as the United States of America, Western Europe, Australia, Japan and Singapore in recent years has resulted in the need for new therapeutic and preventive medical reagents and strategies [1-5]. In addition to pet Felis domesticus and cockroach, house dust mite species, Dermatophagoides pteronyssinus, Dermatophagoides farinae and Blomia tropicalis are the main causative agents of indoor allergen-induced diseases. Blomia tropicalis is geographically localized in tropical and subtropical regions of the world while both Dermatophagoides pteronyssinus and Dermatophagoides farinae are well adapted to temperate, tropical and subtropical areas [1, 2]. The major house dust mite allergens identified in these species, such as Der p 1, Der p 2, Der f 1, Der f 2 and Blo t 5, have a prevalence of more than 60% of IgE reactivity in mite extract skin prick test positive patients [1-3]. Of these mite allergens, Der p 1, Der p 2 and Blo t 5, have been shown in studies based on multi-center skin prick tests to be the major mite allergens in tropical and subtropical countries such as Singapore, Malaysia and Thailand [2]. The study furthermore showed that 20-30% skin prick test positive patients tested positive to multi-major mite allergens, that is, showing skin positive reactions to Der p 1 and/or Der p 2 and/or Blo t 5. Der p 1 and Der p 2 also form the major mite allergens in the temperate, subtropical and tropical geographical regions of the United States of America while Blo t 5 can be found in its tropical and subtropical regions.

Over the past five decades, specific immunotherapy (SIT) based on crude allergen extracts has been shown to be efficacious in treating pollen-induced, cat-induced, and house dust mite-induced allergies [5-9]. However, administration of high doses of the crude allergen extracts has resulted in safety concerns, in particular with regards to the potential risk in triggering life-threatening immediate IgE-mediated anaphylactic reactions. To improve the safety of SIT, there is therefore a need to develop modified low IgE-binding allergens.

A stable recombinant oligomer of Bet v 1 that retains the secondary structural elements of B cell and T cell epitopes of the wild-type allergen has been shown to exhibit reduced allergenic activity in clinical skin test studies on Swedish and French populations [10, 11]. The study also showed that active treatment with the recombinant oligomer resulted in the induction of protective IgG antibodies against new epitopes and a mixed Th2/Th1-like immune response. Another example of a recombinant allergen that is potentially useful in high dose administration SIT is an engineered Der f 2 which, with a disrupted tetra-disulfide bond, has reduced IgE-binding capacity [12-14].

During the last decade, novel vaccines, such as naked DNA, have been shown to be effective in the prophylaxis and treatment of mite allergen-induced asthma in mice [15-16]. International Patent Application No. PCT/SG03/00205 has also shown that the incorporation of a mite allergen boosting strategy, i.e. a DNA prime-protein boost regimen, enhances the specific IgE suppression effect and thereby efficiently inhibit the asthmatic syndrome in mite allergen-sensitized mice.

More recently, a fusion polypeptide comprising two major bee venom allergens, phospholipase A2 and hyaluronidase, was shown to be able to bypass IgE-binding and mast cell or basophil IgE FceRI crosslinking, thereby protecting mice from IgE development [17].

There is a need to provide safer and effective allergens for use in SIT of house dust mite allergen-induced diseases that overcome or at least ameliorate one or more of the disadvantages described above.

There is a need to provide reduced specific IgE-binding capacity chimeric house dust mite allergens for use in SIT against allergic diseases induced by multi-major mite allergens Der p 1, Der p 2 and Blo t 5.

### SUMMARY

The inventors have developed five three-in-one chimeric mite allergens comprising Der p 1, Der p 2 and Blo t 5. Of these, four are GST-fused chimera expressed in Escherichia coli; these are GST-D1proD1B5D2, GST-D1proD1LB5LD2, GST-B5D2D1 and GST-B5D2D1proD1. The fifth, D1proenzD1LB5LD2 was expressed in Chinese hamster ovary (CHO) cells. Subsequent skin prick tests with all five three-in-one chimeric mite allergens showed substantial reduction of mite allergen specific IgE-binding capacity, which indicates the successful generation of hypoallergenic mite allergens. Thus, these three-in-one chimeric mite allergens may be used in the development of safer and effective immunotherapeutic reagents against atopic patients sensitized to multi-major mite allergens Der p 1 and/or Der p 2 and/or Blo t 5.

According to a first aspect of the invention, there is provided a chimeric polypeptide for reducing specific IgE binding to at least two house dust mite allergens in a subject, said chimeric polypeptide comprises an amino acid sequence comprising sequences of said at least two house dust mite allergens and wherein upon exposure to said allergens, said reduced binding causes reduced allergic reaction to said allergens in said subject.

According to a second aspect of the invention, there is provided a pharmaceutical composition comprising a chimeric polypeptide for reducing specific IgE binding to at least two house dust mite allergens in a subject, said chimeric polypeptide comprises an amino acid sequence comprising sequences of said at least two house dust mite allergens and wherein upon exposure to said allergens, said reduced binding causes reduced allergic reaction to said allergens in said subject.

According to a third aspect of the invention, there is provided a vaccine for reducing the severity of an allergic reaction to at least two house dust mite allergens in a subject, the vaccine comprising a chimeric polypeptide capable of reducing specific IgE binding to said at least two house dust mite allergens in said subject, said chimeric polypeptide comprises an amino acid sequence comprising sequences of said at least two house dust mite allergens and wherein upon exposure to said allergens, said reduced binding causes reduced allergic reaction to said allergens in said subject.

According to a fourth aspect of the invention, there is provided a method for generating an immune response against at least two house dust mite allergens in a subject, the method comprising the step of administering to said subject a chimeric polypeptide according to the first aspect, a pharmaceutical composition according to the second aspect or a vaccine according to the third aspect.

According to a fifth aspect of the invention, there is provided a method of desensitizing a subject against house dust mite allergens, the method comprising the step of administering to said subject a chimeric polypeptide according to the first aspect, a pharmaceutical composition according to the second aspect or a vaccine according to the third aspect.

According to a sixth aspect of the invention, there is provided a method of reducing an allergic reaction to house dust mite allergens in a subject, the method comprising the step of administering to said subject a chimeric polypeptide according to the first aspect, a pharmaceutical composition according to the second aspect or a vaccine according to the third aspect.

According to a seventh aspect of the invention, there is provided use of a chimeric polypeptide in the manufacture of a medicament for reducing specific IgE binding to at least two house dust mite allergens in a subject, said chimeric polypeptide comprises an amino acid sequence comprising sequences of said at least two house dust mite allergens and wherein upon exposure to said allergens, said reduced binding is capable of causing reduced allergic reaction to said allergens in said subject.

In one embodiment of the aspects of the invention, the at least two house dust mite allergens are selected from the group consisting of Der p 1, Der p 2 and Blo t 5. The Der p 1 may be include a Der p 1 prodomain sequence.

In another embodiment of the aspects of the invention, the chimeric polypeptide comprises a sequence set forth in SEQ ID NOs. 1, 2, 3, 4 or 5.

Also included within the scope of the invention are fragments of the amino acid sequences of the at least two house dust mite allergens and of the chimeric polypeptide comprising amino acid sequences of said at least two house dust mite allergens. Fragments of sequences set forth in SEQ ID NOs. 1, 2, 3, 4 and 5 are also contemplated as outlined below. Typically, the fragments are allergenic fragments.

In yet another embodiment of the aspects of the invention, there is provided a functional equivalent of the chimeric polypeptide, which retains the reduced specific IgE-binding activity of the reference polypeptide. Preferably, the functional equivalent has at least 60% sequence identity with a polypeptide selected from the group consisting of SEQ ID NOs. 1, 2, 3, 4 and 5. More preferably, the functional equivalent has at least 70%, 80%, 90% or more sequence identity with a polypeptide selected from the group consisting of SEQ ID NOs. 1, 2, 3, 4 and 5.

The subject may be a mammal. In one embodiment, the subject is human. The subject may be exposed to said at least two allergens by active exposure or by passive exposure.

According to an eight aspect of the invention, there is provided a method of producing the chimeric polypeptide of the first aspect, the method comprising the steps of:

(a) providing a gene construct encoding said chimeric polypeptide in a suitable vector;

(b) transforming said vector into a suitable host cell;

(c) culturing said host cell under conditions which permit expression of said chimeric polypeptide; and

(d) collecting and purifying said chimeric polypeptide.

In one embodiment, the vector is pGEX-4T. In an alternative embodiment, the vector is pcDNA3.0. In one embodiment, the host cell may be E. coli or CHO-K1 cells.

### DEFINITIONS

The following words and terms used herein shall have the meaning indicated:

The term “immune response” refers to conditions associated with allergy, inflammation, trauma, immune disorders, or infectious or genetic disease. These conditions may be characterized by expression of various factors, for example, cytokines, chemokines, and other signaling molecules, which may affect cellular and systemic defense systems.

The term “allergen” refers to a substance that is capable of inducing an allergy or an allergic reaction. The allergen may induce an allergy or an allergic reaction by inducing IgE production. The allergen may be a protein or allergenic fragment thereof. Exemplary allergens include but are not limited to pollen, dust-mite droppings, animal dander, mold, fruits, nuts, grasses, antibiotics, bacteria, milk and penicillin.

The term “allergy” refers to the condition of immune hypersensitivity that is greater than normal in an individual who has been exposed to an allergen and has responded with an overproduction of certain immune system components such as immunoglobulin E (IgE) antibodies. Exemplary allergic conditions include eczema, allergic rhinitis or coryza, hay fever, conjunctivitis, bronchial asthma, urticaria (hives) and food allergies, as well as other atopic conditions such as atopic dermatitis, anaphylaxis, drug allergy, angioedema, and allergic conjunctivitis.

The term “allergic reaction” refers to the immediate hypersensitivity response that occurs when a sensitized individual is exposed to an allergen, that is, an IgE-mediated reaction. Such responses are generally associated with the release of histamine from storage cells in tissues. The released histamine binds certain histamine receptors which results in the manifestation of well known allergic symptoms such as sneezing, itching skin, itching eyes, and rhinorrhea.

The term “allergenic” refers to the ability of an allergen to combine, in vivo, with homologous IgE antibodies and thereby induce systemic anaphylaxis or local skin reactions either in passive cutaneous anaphylatic (PCA) reactions or in direct skin test.

The term “hypoallergenic” refers to the decreased ability of an allergen to induce an allergic reaction. This decreased ability to induce an allergic reaction may be due to a decreased ability to combine, in vivo, with homologous IgE antibodies although other mechanisms are also contemplated.

The term “atopic” refers to inherited allergic conditions.

The term “sensitize” refers to the induction of acquired hypersensitivity or of allergy.

The term “desensitize” refers to the reduction or abolition of any form of allergic hypersensitivity or reaction to a specific allergen.

The term “three-in-one” when used in reference to a chimeric mite allergen refers to the presence of three polypeptides or three nucleic acid regions, each encoding one of said three polypeptides, in a single polypeptide or single nucleic acid construct.

The term “individual” when used in reference to an allergen, refers to a single allergen; i.e. not in association with another allergen in the form of a chimeric polypeptide. Individual allergens may be used separately, or they may be combined in a mixture.

The terms “polypeptide”, “peptide” and “protein” refer to any polymer of amino acid residues (dipeptide or greater) linked through peptide bonds or modified peptide bonds and to variants and synthetic analogues of the same. Thus, these terms apply to naturally-occurring amino acid polymers as well as amino acid polymers in which one or more amino acid residues is a synthetic non-naturally occurring amino acid, such as a chemical analogue of a corresponding naturally occurring amino acid. Polypeptides of the present invention include, but are not limited to, products of chemical synthetic procedures, and products produced by recombinant techniques from a prokaryotic or eukaryotic host, including, for example, bacterial, yeast, higher plant, insect and mammalian cells. The polypeptides of the invention may comprise non-peptidic components, such as carbohydrate groups. Carbohydrates and other non-peptidic substituents may be added to a polypeptide by the cell in which the polypeptide is produced, and will vary with the type of cell. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell. Polypeptides are defined herein, in terms of their amino acid backbone structures; substituents such as carbohydrate groups are generally not specified, but may be present nonetheless. In addition, polypeptides of the invention may also include an initial modified methionine residue, in some cases as a result of host-mediated processes. Proteins may be present as monomeric or as multimeric proteins e.g. as dimers (homo or heterodimers) or trimers.

The term “fusion polypeptide” refers to a polypeptide having a plurality of regions, each corresponding to a distinct peptide. Fusion polypeptides can include linkers connecting the regions thereof. Likewise, the term “fusion protein” as used herein, means a protein having a plurality of regions, each corresponding to a distinct peptide. Fusion proteins can include linkers connecting the regions thereof. Typically, for both fusion polypeptides and fusion proteins, while the plurality of regions are unjoined in their native state, they can be joined by their respective amino and carboxyl termini through a peptide linkage to form a single continuous polypeptide or protein. Plurality in this context means at least two. It will be appreciated that the polypeptide or protein components can be joined directly or joined through a linker. Typically, the linker is not part of the sequence of either the fusion partner as outlined below or the target polypeptide/protein. Typically, the linker is a short sequence of amino acids, for example about one to about 20 amino acids. Typically, the linker includes a cleavage recognition site for an enzymatic or chemical cleavage reagent as outlined below so that the fusion partner may be cleaved and purified away from the target polypeptide or protein. The linker may also include additional sequences inserted by one skilled in the art, for example, to provide flexibility to the fusion polypeptide such that the correct formation and/or functioning of the target polypeptide may be achieved, or to provide sufficient spacing between the fusion partner and target polypeptide, or to facilitate cloning. A suitable linker may comprise amino acid repeats such as glycine-serine repeats. A person skilled in the art will be able to design suitable linkers in accordance with the invention. Fusion partners may be used, which may include one or more additional amino acid sequences containing secretory or leader sequences, pro-sequences, or sequences which aid in, for instance detection, expression, separation or purification of the protein or to endow the protein with additional properties as desired such as higher protein stability, for example during recombinant production, or for instance to produce an immunomodulatory response. Examples of potential fusion partners include purification tags, such as a polyhistidine tag, epitope tags (short peptide sequences for which a specific antibody is available) and specific binding proteins; enzymes such as ribonuclease S, glutathione S-transferase (GST), beta-galactosidase, luciferase and hemagglutinin; thioredoxin; a secretion signal peptide and a label, which may be, for instance, bioactive, radioactive, enzymatic or fluorescent, or an antibody.

The term “fusion genes” refers to a polynucleotide comprising a plurality of regions. Plurality in this context means at least two.

The term “wild-type” refers to a gene or gene product which has the characteristics of that gene or gene product when isolated from a naturally occurring source. A wild-type gene may also be one that is most frequently observed in a population.

The term “variant” refers to a polynucleotide or polypeptide that differs from a parent polynucleotide or polypeptide respectively, but retains essential properties. A typical variant of a polynucleotide differs in nucleotide sequence from another, parent polynucleotide. Changes in the nucleotide sequence of the variant may or may not alter the amino acid sequence of a polypeptide encoded by the parent polynucleotide. Nucleotide changes may result in amino acid substitutions, additions, deletions, fusions and truncations in the polypeptide encoded by the parent sequence, as discussed below. A typical variant of a polypeptide differs in amino acid sequence from another, parent polypeptide. Generally, differences are limited so that the sequences of the parent polypeptide and the variant are closely similar overall and, in many regions, identical. A variant and parent polypeptide may differ in amino acid sequence by one or more substitutions, additions and deletions in any combination. A substituted or inserted amino acid residue may or may not be one encoded by the genetic code.

The term “fragment” includes a nucleic acid or polypeptide molecule that encodes a constituent or is a constituent of a particular nucleic acid or polypeptide or variant thereof. In terms of the polypeptide, the fragment possesses qualitative biological activity in common with the polypeptide in question. The fragment may be physically derived from the full-length nucleic acid or polypeptide or alternatively may be synthesized by some other means, for example chemical synthesis. The fragments should comprise at least n consecutive amino acids from the parent sequence and, depending on the particular sequence, n preferably is 5 or more (for example, 5, 6, 7, 8, 9, 10, 12, 15, 17, 20, 30, 40, 50, 60, 70 or 80 or more). Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. Additionally, several fragments may be comprised within a single larger polypeptide. In terms of the nucleic acid, the fragment does not necessarily need to encode polypeptides which retain biological activity, for example, hybridisation probes or PCR primers.

The term “functional equivalent” refers to a polypeptide that retains a biological activity of the parent polypeptide. Typically, the biological activity is allergenicity. The functionally-equivalent polypeptide may be homologous to the parent polypeptide or to a natural biological variant or an analogue thereof. Functional equivalents of the polypeptides may also include polypeptides in which relatively short stretches have a high degree of homology (at least 60%, 70%, 80%, 85%, 90%, 92%, 95%, 97% or more) with the parent polypeptide even though the overall homology between the two polypeptides may be much less. This is because important recognition or binding sites may be shared even when the general architecture of the polypeptide is different.

The term “chimeric” is used herein to describe the state of being a chimera. A “chimera” refers to a construct comprising polypeptide or gene sequences not typically found in association with each other. The polypeptide or gene sequences in the chimera may be from different origins. For example, a chimera may comprise a combination of a polypeptide sequence from one species with a polypeptide sequence from another species, a combination of wild type polypeptide with a recombinant polypeptide, a combination of wild type sequence and patient derived sequence. It will be noted that the chimera may comprise any combination of polypeptides or gene sequences not typically found in association with each other. In addition to the examples mentioned above, the chimera may thus comprise multiple (i.e. two or more) polypeptides or sequences from a single species, strain or organism. The chimera may thus include any combination of polypeptides or gene sequences from a single organism or any combination from multiple different organisms or both. The chimeric polypeptide or gene may or may not be humanized. It should be noted that when reference is made to a chimeric polypeptide or a chimeric gene, this term also includes mutant polypeptides or mutant genes that still essentially have the same biological function or encode a polypeptide having essentially the same biological function, respectively. It should be clear that any method known in the art to develop chimeric polypeptide and gene constructs may be used.

The term “humanized” means that at least a portion of the framework regions of an immunoglobulin or engineered antibody construct is derived from human immunoglobulin sequences.

The term “primer” refers to a single-stranded oligonucleotide capable of acting as a point of initiation of template-directed DNA synthesis. The precise length of a primer will vary according to the particular application, but typically ranges from 15 to 30 nucleotides. A primer need not reflect the exact sequence of the template but must be sufficiently complementary to hybridize to the template. An appropriate primer length and sequence may readily be determined by one of ordinary skill in the art.

The term “antigen” refers to any foreign substance that is bound by a specific antibody or specific lymphocyte. An antigen may be capable of inducing an immune response, i.e. an immunogen. An antigen may also be capable of inducing an allergic reaction, i.e. an allergen. Alternatively, an antigen may be a pathogen.

The term “backbone”, when used in reference to the chimeric mite allergens of the invention, refers to the sequence of amino acid residues in the chimeric mite allergen that contains the N-terminal leader sequence.

The term “induce” and grammatical variants thereof refers to the triggering of an immune response by exposure to one or more antigens and/or allergens. The term also includes the triggering of an immune response by a vaccine or a set of vaccines.

The terms “coupled to”, “coupling”, “bind to”, “binding” or grammatical variants thereof refer to any type of physical association between two components. The two components may be for example, the polypeptide sequence of one mite allergen and the polypeptide sequence of another mite allergen, the polypeptide sequence of a mite allergen and a N-terminal leader sequence, a mite allergen and a fusion partner as described above, or a mite allergen and an IgE antibody. The association may be direct or may be indirect, for example through the use of one or more linkers as described above. The association may also be covalent or non-covalent. Coupling may be achieved using any chemical, biochemical, enzymatic or genetic coupling known to those skilled in the art.

The term “exposed to” refers to either the active step of contacting the subject with an antigen or the passive exposure of the subject to the antigen in vivo. The antigen may be an allergen. Methods for the active exposure of a subject to an antigen are well-known in the art. In general, an antigen may be administered directly to the subject by any means such as intravenous, intramuscular, oral, transdermal, mucosal, intranasal, intratracheal, or subcutaneous administration. The antigen may also be administered systemically or locally. A subject is passively exposed to an antigen if an antigen becomes available for exposure to the immune cells in the body. A subject may be passively exposed to an antigen, for instance, by entry of a foreign pathogen into the body or by the development of a tumor cell expressing a foreign antigen on its surface.

The term “subject” refers to any animal, including mammals such as humans.

The term “immunotherapy” refers to a treatment regimen based on activation of a antigen-specific immune response. Examples of immunotherapy include desensitization with a specific allergen, administration of vaccines and the charging of dendritic cells with EBNA-1 antigen, preferably with a stimulatory cytokine such as GM-C SF or Flt3 ligand ex vivo or in vivo.

The term “treatment” includes any and all uses which remedy or ameliorate a disease state or symptoms, prevent the establishment of disease, or otherwise prevent, hinder, retard, or reverse the progression of disease or other undesirable symptoms in any way whatsoever. Treatment may be effected prophylactically or therapeutically. Treatment may entail treatment with a single agent or with a combination (more than two) of agents. An “agent” is used herein broadly to refer to, for example, a compound such as the chimeric mite allergens of the invention.

The term “therapeutically effective amount” includes a non-toxic but sufficient amount of a compound to provide the desired therapeutic effect. The exact amount required will vary from subject to subject depending on factors such as the species being treated, the age and general condition of the subject, the severity of the condition being treated, the particular compound being administered and the mode of administration and so forth. Thus, it is not possible to specify an exact “effective amount”. However, for any given case, an appropriate “effective amount” may be determined by one of ordinary skill in the art using only routine experimentation.

The term “vaccine” includes an agent which may be used to stimulate the immune system of an animal. In this way, immune protection may be provided against an antigen not recognized as a self-antigen by the immune system. Typically, the agent is a polypeptide such as the chimeric mite allergen of the invention. The vaccine may also be a DNA or an RNA. The DNA or RNA may be delivered by means of a recombinant vector for expression of chimeric polypeptide of the invention. In some instances, the vector may be a virus, for example a retrovirus or a lentivirus.

The term “expression” as used herein refers interchangeably to expression of a gene or gene product, including the encoded polypeptide.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DISCLOSURE OF EMBODIMENTS

Exemplary, non-limiting embodiments of chimeric polypeptides, compositions containing said chimeric polypeptides and methods for their production and use will now be disclosed.

The first aspect of the invention provides a chimeric polypeptide for reducing specific IgE binding to at least two house dust mite allergens in a subject, said chimeric polypeptide comprises an amino acid sequence comprising sequences of said at least two house dust mite allergens and wherein upon exposure to said allergens, said reduced binding causes reduced allergic reaction to said allergens in said subject.

In one embodiment, the chimeric polypeptide comprises two house dust mite allergens. In another embodiment, the chimeric polypeptide comprises three house dust mite allergens. In yet another embodiment, the chimeric polypeptide comprises four, or five, or six or more house dust mite allergens. A person skilled in the art would appreciate that where a chimeric polypeptide comprising three or more allergens has been shown to be effective against said three or more allergens, then a chimeric polypeptide comprising any two of the three or more allergens would also be effective against said two of the three or more allergens.

In one embodiment, the at least two house dust mite allergens may be selected from the group consisting of Der p 1, Der p 2 and Blo t 5. For patients sensitized by Der p 1 and Der p 2, a suitable chimeric polypeptide may comprise Der p 1 and Der p 2, or the chimeric polypeptide may comprise Der p 1, Der p 2 and Blo t 5. For patients sensitized by Der p 1 and Blo t 5, a suitable chimeric polypeptide may comprise Der p 1 and Blo t 5, or the chimeric polypeptide may comprise Der p 1, Der p 2 and Blo t 5. For patients sensitized by Der p 2 and Blo t 5, a suitable chimeric polypeptide may comprise Der p 2 and Blo t 5, or the chimeric polypeptide may comprise Der p 1, Der p 2 and Blo t 5. In some embodiments, Der p 1 may include a Der p 1 prodomain sequence.

In still further embodiments, any other house dust mite allergens may be used. Such other house dust mite allergens may be from Dermatophagoides species (for example D. pteronyssinus and D. farinae) or Blomia species (Blomia tropicalis), or they may be from other house dust mite species, such as Euroglyphus (for example Euroglyphus maynei). Typically, such other house dust mite allergens are those which display cross-reactivity with Der p 1, Der p 2 and Blo t 5. For example, the highly homologous sequences of mite allergens within the Dermatophagoides species (up to 90%) results in a high degree of cross-reactivity between said homologous allergens and hence, other Dermatophagoides mite allergens such as Der f 1, Der f 2, Der f 3, Der f 9, Der f 11, Der f 13, Der f 14, Der f 15 are also included within the scope of the invention. Likewise, cross-reactivity is also known to exist between allergens of different house dust mite species. For example, Blo t 5, a homologue of Der p 5, has been reported to be cross-reactive to it, while Blo t 10 and Der p 10, which share 95% amino acid identity, are also highly cross-reactive.

Where appropriate, the chimeric polypeptides may include fusion partners as outlined above, for example GST for purification purposes, and/or linker sequences as outlined above, for example glycine/serine linkers.

The mite allergens, fusion partners and/or linkers of the chimeric polypeptides may be organized in any order. Preferably, the mite allergens, fusion partners and/or linkers are organized in the orders as set forth in SEQ ID NOs. 1, 2, 3, 4 or 5.

Also included within the scope of the invention are fragments of the amino acid sequences of the at least two house dust mite allergens and of the chimeric polypeptide comprising amino acid sequences of said at least two house dust mite allergens. Fragments of sequences set forth in SEQ ID NOs. 1, 2, 3, 4 and 5 are also contemplated. Typically, the fragments are antigenic fragments. The fragments may contain single or multiple amino acid deletions from either terminus of the chimeric polypeptide or from internal stretches of the primary amino acid sequence. As outlined above, the fragments may comprise at least n amino acids from the parent polypeptide sequence, where n is preferably 5 or more. Hence, a fragment of SEQ ID No. 1 may comprise about 5 to about 467 amino acid residues while a fragment of SEQ ID No. 2 may comprise about 5 to about 547 amino acid residues. Similarly, a fragment of SEQ ID No. 3 may comprise about 5 to about 547 amino acid residues, a fragment of SEQ ID No. 4 may comprise about 5 to about 571 amino acid residues and a fragment of SEQ ID No. 5 may comprise about 5 to about 567 amino acid residues In one embodiment, the fragment may retain the reduced specific IgE-binding activity of the parent polypeptide.

Hence, the invention also includes functional equivalents of the chimeric polypeptides, which retains the reduced specific IgE-binding activity of the reference polypeptide. Preferably, the functional equivalent has at least 60% sequence identity with a polypeptide selected from the group consisting of SEQ ID NOs. 1, 2, 3, 4 and 5. More preferably, the functional equivalent has at least 70%, 80%, 90% or more sequence identity with a polypeptide selected from the group consisting of SEQ ID NOs. 1, 2, 3, 4 and 5. Fragments and variants of the functional equivalents of the chimeric polypeptides are also included within the scope of the present invention.

Upon exposure to any one or any two or all three of the mite allergens, a subject desensitized with a chimeric polypeptide comprising at least two of the appropriate mite allergens will have a reduced allergic reaction to those allergens. For example, a subject that has been desensitized with a chimeric polypeptide comprising Der p 1 and Der p 2 will have a reduced allergic reaction upon exposure to Der p 1 alone, or to Der p 2 alone, or to Der p 1 and Der p 2. Likewise, a subject that has been desensitized with a chimeric polypeptide comprising Der p 1, Der p 2 and Blo t 5 will have a reduced allergic reaction upon exposure to Der p 1 alone, or Der p 2 alone, or Blo t 5 alone, or Der p 1 and Der p 2, or Der p 1 and Blo t 5, or Der p 2 and Blo t 5, or Der p 1, Der p 2 and Blo t 5. All other possible combinations are also contemplated.

The reduced allergic reaction may be due to the reduced specific binding of the allergen to the IgE. The reduced allergic reaction may also be due to the attenuation of histamine release and/or to increased production of IL-10 and/or to inhibition of T-helper cell cytokine production.

The subject may be a mammal. In one embodiment, the subject is human. The subject may be exposed to said at least two allergens by active exposure or by passive exposure as outlined above.

The second aspect of the invention provides a pharmaceutical composition comprising a chimeric polypeptide of the first aspect. The composition may comprise one or more of said chimeric polypeptides. In general, suitable compositions may be prepared according to methods which are known to those of ordinary skill in the art and accordingly may include a pharmaceutically acceptable carrier, diluent and/or adjuvant.

The phrase “pharmaceutically acceptable” refers to molecular entities and compositions that are physiologically tolerable and do not typically produce an allergic or similar untoward reaction, such as gastric upset, dizziness and the like, when administered to the subject. The carriers, diluents and adjuvants must also be “acceptable” in terms of being compatible with the other ingredients of the composition.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

These compositions may be administered by any standard routes. For example, the compositions of the invention may be in a form suitable for administration by injection; in the form of a formulation suitable for oral ingestion such as capsules, tablets, caplets, or elixirs; in the form of an ointment, cream or lotion suitable for topical administration; in a form suitable for delivery as an eye drop; in an aerosol form suitable for administration by inhalation such as by intranasal inhalation or oral inhalation; in a form suitable for parenteral administration, that is, by subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

A third aspect of the invention provides a vaccine for reducing the severity of an allergic reaction to at least two house dust mite allergens in a subject, the vaccine comprising one or more of the chimeric polypeptides of the first aspect.

The vaccine may comprise a pharmaceutically acceptable carrier and/or diluent as outlined above, and/or an adjuvant. The adjuvant is a substance that increases the immunological response of the subject to the vaccine. Suitable adjuvants include, but are not limited to, aluminum hydroxide (alum), immunostimulating complexes (ISCOMS), non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

A fourth aspect of the invention provides a method for generating an immune response against at least two house dust mite allergens in a subject, the method comprising the step of administering to said subject a chimeric polypeptide according to the first aspect, a pharmaceutical composition according to the second aspect or a vaccine according to the third aspect. The immune response may be an allergic reaction, for example, histamine release, production of IL-10 and/or subsequent inhibition of T-helper cell cytokine production.

A fifth aspect of the invention provides a method of desensitizing a subject against house dust mite allergens, the method comprising the step of administering to said subject a chimeric polypeptide according to the first aspect, a pharmaceutical composition according to the second aspect or a vaccine according to the third aspect.

A sixth aspect of the invention provides a method of reducing an allergic reaction to house dust mite allergens in a subject, the method comprising the step of administering to said subject a chimeric polypeptide according to the first aspect, a pharmaceutical composition according to the second aspect or a vaccine according to the third aspect.

It will be appreciated that the route and dosage of administration in the methods of the fourth, fifth and sixth aspects will be readily apparent to one skilled in the art and may, where appropriate, be readily determined through routine experimentation. For example, the route of administration may be any standard route such as by the parenteral (e.g. intravenous, intraspinal, subcutaneous or intramuscular), oral or topical route. The therapeutically effective dose level for any particular subject will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One or more doses of the chimeric polypeptide according to the first aspect, a pharmaceutical composition according to the second aspect or a vaccine according to the third aspect may be administered. Typically, the dose is gradually increased until a dose adequate to control symptoms (maintenance dose) is reached.

A seventh aspect of the invention provides use of a chimeric polypeptide in the manufacture of a medicament for reducing specific IgE binding to at least two house dust mite allergens in a subject, said chimeric polypeptide comprises an amino acid sequence comprising sequences of said at least two house dust mite allergens and wherein upon exposure to said allergens, said reduced binding is capable of causing reduced allergic reaction to said allergens in said subject.

An eight aspect of the invention provides a method of producing the chimeric polypeptide of the first aspect, the method comprising the steps of:

(a) providing a gene construct encoding said chimeric polypeptide in a suitable vector;

(b) transforming said vector into a suitable host cell;

(c) culturing said host cell under conditions which permit expression of said chimeric polypeptide; and

(d) collecting and purifying said chimeric polypeptide.

The gene constructs may be designed based on known sequences and generated using methods known to one of ordinary skill in the art such as PCR. The methods and reagents for use in PCR amplification reactions, restriction enzyme digestion and subsequent fragment resolution, and nucleic acid sequencing are well known to those skilled in the art. In each case, suitable protocols and reagents will largely depend on individual circumstances. Guidance may be obtained from a variety of sources, such as for example Sambrook et al., Molecular Cloning: A Laboratory Manual, Cold Spring Harbor, N.Y., 1989, and Ausubel et al., Current Protocols in Molecular Biology, Greene Publ. Assoc. and Wiley-Intersciences, 1992. A person skilled in the art would readily appreciate that various parameters of these procedures may be altered without affecting the ability to achieve the desired product. For example, in the case of PCR amplification, the salt concentration may be varied. Similarly, the amount of DNA used as a template may also be varied depending on the amount of DNA available or the optimal amount of template required for efficient amplification.

Any suitable vector may be used and selection of such suitable vectors may be readily determined by one of ordinary skill in the art. In one embodiment, the vector is pGEX-4T. In an alternative embodiment, the vector is pcDNA3.0.

The expression vector construct may be introduced into an appropriate host cell through conventional methods such bacteriophage or viral infection, electroporation, heat shock, lipofection, and particle bombardment. Host cells such as Gram-positive bacteria belonging to the genus Bacillus and Gram-negative bacteria such as Escherichia coli may be used. In one embodiment, mammalian host cells are used, for example CHO-K1 cells. Selected strains of host cells are inoculated in a medium containing an assimilable carbon source, a nitrogen source and essential nutrients, and are cultured through conventional fermentation methods. Conditions of the culture may be readily determined through routine experimentation. Collection and purification of the chimeric polypeptide from the thus-obtained culture broth can be performed according to conventional methods applicable to the collection and purification of common proteins. For example, cells are separated from the culture broth by centrifugation or filtration, and the chimeric polypeptide can be obtained from the supernatant through conventional purification procedures, examples of which include: fractionation on an ion-exchange column; ethanol precipitation; affinity chromatography; ultracentrifugation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind polyhistidine-tagged forms of polypeptides. Various methods of protein purification may be employed and such methods are known in the art and described for example in Deutscher, Methods in Enzymology, 182 (1990) and Scopes, Protein Purification: Principles and Practice, Springer-Verlag, New York (1982). The purification step(s) selected will depend, for example, on the nature of the production process used and the chimeric polypeptide produced.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1. Schematic representation of GST-fused three-in-one chimeric mite allergens.

FIG. 2. Polypeptide sequences of the three-in-one chimeric mite allergens GST-cleaved B5D2D1 (SEQ ID NO. 1), GST-cleaved B5D2D1proD1 (SEQ ID NO. 2), GST-cleaved D1ProD1B5D2 (SEQ ID NO. 3) and GST-cleaved D1proD1LB5LD2 (SEQ ID NO. 4). The underlined residues indicate the sequence derived from the thrombin cleavage site. The sizes of chimeric mite allergens GST-cleaved B5D2D1, GST-cleaved B5D2D1proD1, GST-cleaved D1ProD1B5D2 and GST-cleaved D1proD1LB5LD2 are 468 residues, 548 residues, 548 residues and 572 residues, respectively. Bold sequences denote the glycine/serine linker.

FIG. 3. SDS-PAGE gel analysis of purified GST-cleaved three-in-one chimeric mite allergens. Purified three-in-one chimeric mite allergens (4 mg) were digested with thrombin (0.25 U) for 3 hours at room temperature and then resolved on 7.5% SDS-PAGE gel. Lanes a, b, c, d and M denote GST-D1proD1B5D2, GST-D1proD1LB5LD2, GST-B5D2D1, GST-B5D2D1proD1, and the polypeptide size marker, respectively.

FIG. 4. Skin prick test reactions of atopic subjects to GST-fused three-in-one chimeric mite allergens. Chimerics A, B, C and D represent the three-in-one chimeric allergens GST-D1proD1B5D2, GST-D1proD1LB5LD2, GST-B5D2D1, GST-B5D2D1proD1. yDer p 1, yDer p 2, yBlo t 5, Der p mite extract and Blo t mite extract are also included in this study. (ND: Not Done).

FIG. 5. Cytokine profiling of PBMC from one normal (N3) and one atopic (A) subject when co-cultured with or without individual or chimeric allergen (Der p 1, Der p 2, Blo t 5 and chimeric GST-B5D2D1proD1).

FIG. 6. Generation of a codon optimized chimeric gene VZVproenz-Dp1-Bt5-Dp2 (D1proenzD1LB5LD2). Partially overlapping primers C1, C2, C3, C4, C5, and C6 were used to generate this chimeric gene by PCR, with a linker (L) incorporated into these primers.

FIG. 7. Polypeptide sequence of the three-in-one chimeric mite allergen D1proD1LB5LD2 (SEQ ID NO. 5). The size of CHO-K1-expressed three-in-one chimeric mite allergen D1proD1LB5LD2 is 568 residues. The bold sequence denotes the glycine/serine linker.

FIG. 8. SDS-PAGE of affinity purification of CHO-K1-expressed three-in-one chimeric D1proenzD1LB5LD2. A total of 20 μl from each eluted fraction was resolved on a 10% Tris-Tricine SDS-PAGE for Coomassie staining (A) and 200 ng chimeric polypeptide for Western analysis (B). The immunoblot was probed with mAb for Der p 1 (4C1) (5000× dilution), Blo t 5 (4A7) (10,000× dilution) and Der p 2 (C5) (10,000× dilution), respectively, overnight at 4° C., followed by a 1 h incubation at room temperature with biotinylated anti-mouse Ig (5000× dilution) and detection with conjugated Extravidin peroxidase (5000× dilution), respectively. Lane M: Polypeptide molecular weight marker (BioRAD); (A) Lanes 1-9: Eluted fractions 1 to 9. The arrow indicates the affinity purified chimeric polypeptide. (B) Lanes: M) Biotinylatedpolypeptide molecular weight marker; a) yDer p 1,100 ng; c) yBlo t 5, 100 ng; e) yDer p 2, 100 ng; (b, d, f) chimeric polypeptide 200 ng.

FIG. 9. Skin prick tests (wheal and/or erythema size) on 15 atopic subjects (A-L are adults and M-O are children) which are either positive to one or two of the allergens or to all three allergens. Subjects B, M (sensitized to all three allergens Der p 1, Der p 2 and Blo t 5) and A, O (sensitized to only Der p 2 and Blo t 5) tested positive in skin prick tests against the CHO-K1-expressed three-in-one chimeric mite allergen with an overall of 26.6% positive reactivity in this panel of atopic subjects.

FIG. 10. Histamine release profiles of two atopic subjects (A and B) induced by a three-in-one chimeric polypeptide or a mixture of three individual allergens (Der p 1, Der p 2 and Blo t 5). Whole blood from two atopic subjects was incubated with different molar concentrations of either the CHO-K1-expressed chimeric polypeptide or a mixture of three individual allergens. Histamine release (expressed in ng/ml) was performed and assayed using the Histamine-Release and Histamine ELISA kit from IBL.

FIG. 11. Adsorption ELISA assay. Sera from two sensitized subjects (1 and 2) were pre-absorbed with various concentrations of CHO-K1-expressed chimeric polypeptide before incubation with (A) yDer p 1, (B) yDer p 2 or (C) yBlo t 5 coated plates. Subjects 1 and 2 have high Der p 2- and Blo t 5-specific serum IgE titers, respectively.

FIG. 12. Cytokine profiling of PBMC from two normal (N1 and N2) and three atopic (A, B and C) subjects when co-cultured with CHO-K1-expressed three-in-one chimeric polypeptide, a mixture of the three individual allergens (nDer p 1, yDer p 2 and yBlo t 5) or the medium alone. Appropriate molar (pM) ratios were used in determining the concentration for the CHO-K1-expressed three-in-one chimeric polypeptide and the three individual allergen mixtures.

### BEST MODE

Non-limiting examples of the invention, including the best mode, and a comparative example will be further described in greater detail by reference to specific Examples, which should not be construed as in any way limiting the scope of the invention.

### EXAMPLE1

A. Production of Three-in-One Chimeric Mite Allergens as GST Fusion Polypeptides in E. coli

Design of Chimeric Constructs

In contrast to recombinant Blo t 5 [20], recombinant Der p 1 and Der p 2 expressed as GST fusion polypeptides in E. coli are insoluble and resistant to thrombin cleavage. Accordingly, GST-Blo t 5 was selected as one of the two backbone constructs for the design of the three-in-one chimeric mite allergen fusion genes.

Further, as the Der p 1 prodomain is known to play a role in chaperoning the mature Der p 1 [25], the Der p 1 prodomain was coupled to the mature Der p 1 in the three-in-one chimeric allergens to facilitate proper folding of the polypeptide and thereby improve solubility. This resulted in backbone constructs of GST-Der p 1 prodomain.

In addition, the mite allergens (Der p 1, Der p 2, and Blo t 5) were coupled via glycine/serine linkers to confer flexibility and stability to each allergen moiety, which in turn ensures that the proper folding and solubility of the chimeric mite allergens are maintained.

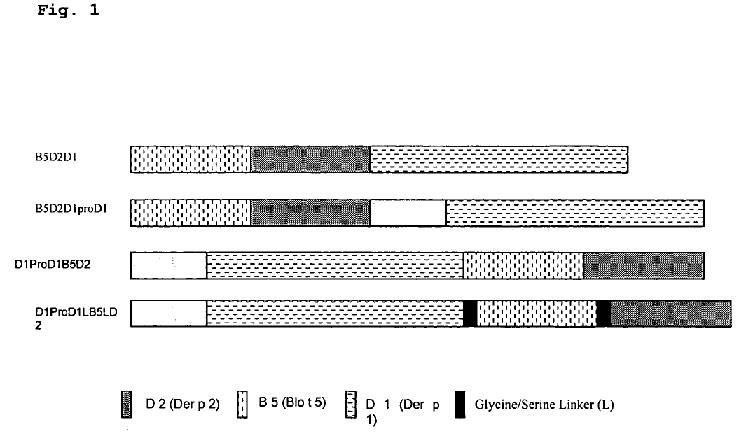

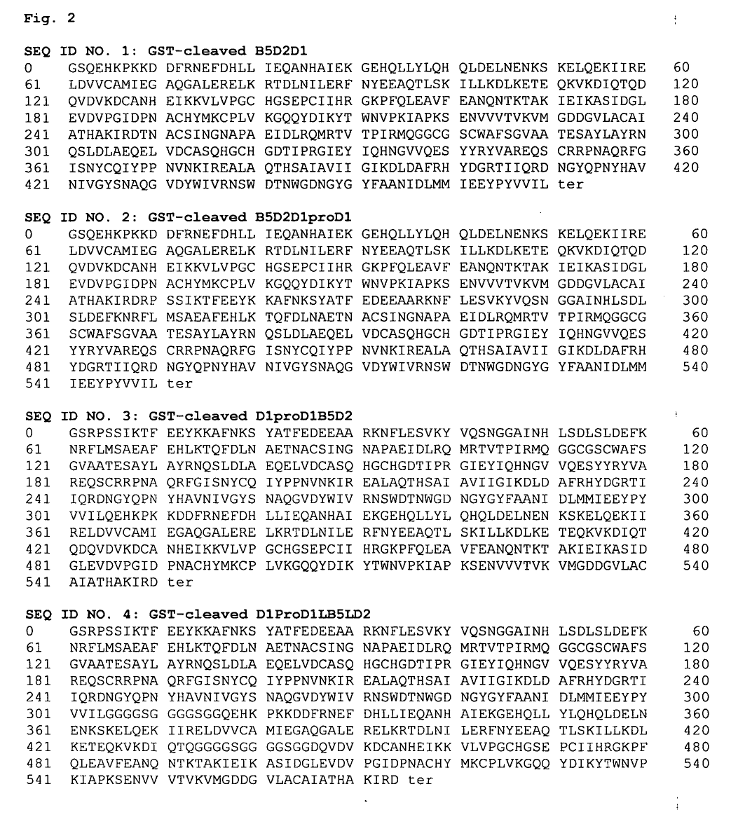

Thus, four three-in-one chimeric mite allergen gene constructs, were produced; these were D1proD1B5D2, D1proD1LB5LD2, B5D2D1 and B5D2D1proD1. The gene organization of the four three-in-one chimeric mite allergen gene constructs are shown in FIG. 1 while the corresponding amino acid sequences are shown in FIG. 2.

Materials and Methods

Generation of GST-Fused Expression Constructs

The sequences encoding the three-in-one chimeric mite allergens for mature Der p 1, Der p 2 and Blo t 5 were amplified by PCR using specific overlapping oligonucleotide primers based on known sequences. The resultant PCR fragments were then cloned into the pCR vector using the TA cloning kit (Gibco-BRL-Invitrogen) and the sequences verified using ABI PRISM™ 377 DNA sequencer using ABI PRISM® BigDye™ Terminators v 3.0 Cycle Sequencing Kit. The verified chimeric sequences were subcloned into an expression vector of the Glutathione S-Transferase (GST) Gene Fusion System, pGEX-4T (Amersham Phamacia Biotech), and transformed into DH5α host cells. Four chimeric mite allergen gene constructs were produced, the gene organizations of which are schematically shown in FIG. 1. The vectors containing the chimeric mite allergen gene constructs were pGEX-4T-B5D2D1, pGEX-4T-B5D2D1proD1, pGEX-4T-D1proD1B5D2, and pGEX-4T-D1proD1LB5LD2.

Screening of E. coli Clones Expressing GST-Fused Three-in-One Chimeric Mite Allergens

To achieve a high yield of the chimeric mite allergens in the bacterial expression system, BL21 (DE3), a protease-deficient strain of E. coli was used as the host for optimizing the production of the chimeric mite allergens. Screening for high expression BL21 (DE3) transformant clones was performed as described by Chua et al [18]. An overnight culture of each transformant clone harbouring the desired plasmid (as verified with DNA sequencing) was diluted 1:50 in 3 ml fresh LB medium at 37° C. with rigorous shaking until the OD600 reaches 0.5 (˜2 to 3 h). Isopropyl-α-dthiogalactopyranoside was added to obtain a final concentration of 0.1M for a 3-hour induction. Cell pellets resuspended in 0.5 ml TBS (10 mM Tris, 150 mM NaCl, pH 7.5) containing 1 mM PMSF and DNAse I (final concentration=20 μg/ml) were disrupted by sonication on ice using Soniprep 150 (MSE). The cell lysates were removed by micro-centrifugation at 12000 rpm for 10 minutes at 4° C. and 10 μl clear supernatants were used for Tricine-SDS-PAGE analysis. Transformant clones with the highest chimeric mite allergen expression levels were selected for further scale-up production.

Expression and Purification of the GST-Fused Three-in-one Chimeric Mite Allergens

The chimeric mite allergens were expressed as a glutathione S-transferase (GST) fusion polypeptide in E. coli (BL21 strain) and purified using a glutathione Sepharose column (Sigma, USA) as described by Chua et al. [18]. Overnight cell cultures were added to freshly prepared LB broth (1:50/v:v) and expanded at 37° C. with shaking (250 rpm) for 3 hours (O.D˜0/6). Induction was carried out with addition of 1 mM isopropyl-a-d-thiogalactopyranoside (Calbiochem-Novabiochem, Darmstadt, Germany) at 30° C. with shaking (250 rpm) for 3 hours. The expressed products were analyzed using 7.5% SDS-PAGE.

Allergenicity Determination by Skin Prick Tests

The skin of the forearm's volar was pricked with a disposable lancet in the presence of an allergen droplet. The prick test was considered positive when the wheal diameter was 3×3 mm larger than the negative control. Glycerol-buffer and GST (25 μg/ml), and 1 mg/ml of histamine were used as negative and positive controls, respectively. 25 μg/ml of purified allergen was used in this study.

Results and Discussion

Expression and Production of the GST-Fused Three-in-one Chimeric Mite Allergens

The organization of the genes encoding Der p 1, Der p 2 and Blo t 5 in the three-in-one chimeric mite allergens is shown in FIG. 1. Two different types of backbones, Blo t 5 or Der p 1 prodomain as the N-terminal leader, were used for the four three-in-one chimeric mite allergens. These resulted in the production of GST-cleaved B5D2D1, GST-cleaved B5D2D1proD1, GST-cleaved D1ProD1LB5LD2 and GST-cleaved D1proD1B5D2. The amino acid sequences of the GST-cleaved three-in-one chimeric allergens are shown in FIG. 2.

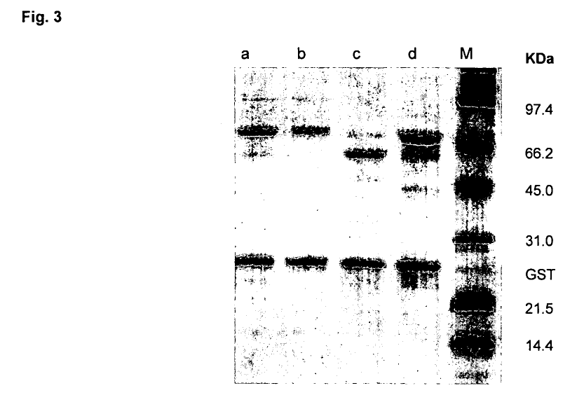

In this example, E. coli GST Gene Fusion System was employed for large scale expression and production of the three-in-one chimeric mite allergens. Despite comprising three non-evolutionary related genes that encode polypeptides with different topological structures and biological functions, a substantial amount of soluble three-in-one chimeric mite allergens were produced under optimal expression conditions. Under the same expression conditions, higher amounts of the soluble three-in-one chimeric mite allergens with Blo t 5 as the N-terminal leader were produced. The results also show that all four three-in-one chimeric mite allergens, GST-D1proD1B5D2, GST-D1proD1LB5LD2, GST-B5D2D1 and GST-B5D2D1proD1, were cleavable upon thrombin digestion. However, upon incubation with thrombin for 3 hours at room temperature, both GST-B5D2D1 and GST-B5D2D1proD1 were shown to be fully cleaved while GST-D1proD1B5D2 and GST-D1proD1LB5LD2 were shown to be less susceptible to thrombin digestion with some undigested polypeptides remaining in the digestion mixture (FIG. 3).

Comparison of the yields of the four three-in-one chimeric mite allergens purified from the expression lysate also showed that expression was highest for GST-B5D2D1ProD1; a yield of about 200 mg soluble polypeptide from one gram of E. coli was obtained. This yield was double that of GST-D1ProD1LB5LD2 and GST-B5D2D1, and triple that of GST-D1ProD1B5D2.

Clinical Evaluation of Three-in-One Chimeric Mite Allergens

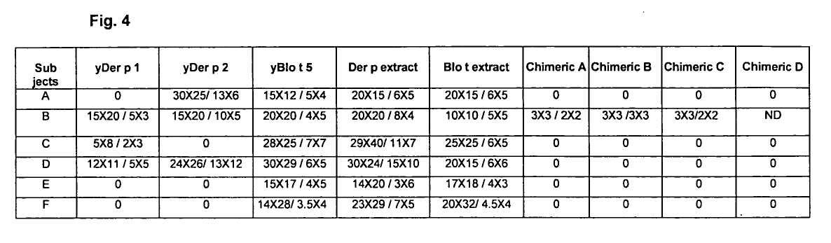

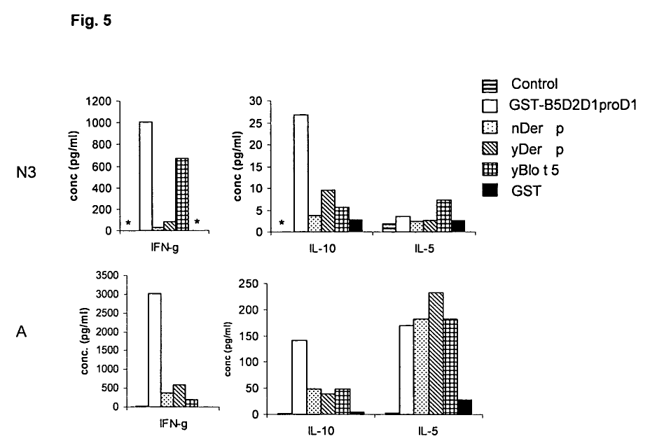

Six atopic subjects were selected for the evaluation of the four three-in-one chimeric mite allergens using skin prick test. Among the tested subjects, only subject #2 reacted to all four three-in-one chimeric mite allergens. None of the other subjects showed any responses to the skin prick test (FIG. 4). Thus, the results show that all four three-in-one chimeric mite allergens are hypoallergenic compared to the individual wild-type mite allergens. The results of the cytokine profiling of peripheral blood mononuclear cells (PBMC) is shown in FIG. 5.

### EXAMPLE2

B. Production of Humanized Three-in-One Chimeric Mite Allergen in CHO-K1 Cells

Materials and Methods

Generation of Codon Optimized/Humanized Three-in-One Chimeric Mite Allergen vzv-D1proenzD1LB5LD2 Gene

Codon optimized pro-enzyme-Der p 1, Der p 2 and Blo t 5 genes were respectively generated based on the codon preference of highly expressed human genes. These genes were synthesized by PCR using sets of partially overlapping oligonucleotide primers and subsequently cloned into TA-TOPO vector to facilitate DNA sequencing. An efficient leader peptide of the varicella-zoster virus (VZV) glycoprotein E which facilitates secretion was tagged in-frame to the N-terminal of the synthetic genes by PCR before cloning into the mammalian expression vector pcDNA3.0. [23, 24, 25]. All PCR reactions were performed using the Expand High Fidelity DNA Polymerase (Boehringer).

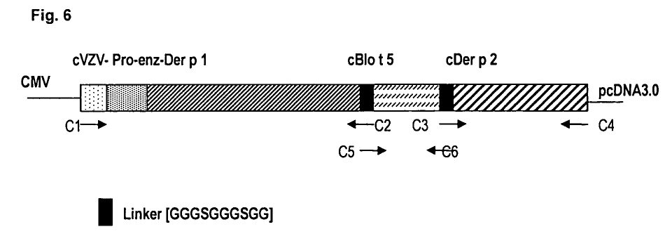

PCR fragments of pro-enzyme-Der p 1, Der p 2 and Blo t 5 were respectively generated using the following oligonucleotide primer pairs; C1 and C2, C3 and C4, C5 and C6, respectively (FIG. 6). To facilitate subcloning, a BamHI site and a XbaI site were respectively included in the primers C1 and C4. In addition, spacer sequences were incorporated into the junction of the fusion genes by insertion of the following oligonucleotides:

First insert:
GGA GGG GGC TCC GGA GGG GGC TCC GGA GGG
Second insert:
GGC GGC GGG AGC GGC GGC GGG AGC GGC GGC

Primers C2 and C5 contain the sequence of the first insert while primers C3 and C6 contain the sequence of the second insert.

Codon optimized Blo t 5 and Der p 2 were fused together by PCR. A mixture of the codon optimized Blo t 5 and Der p 2 PCR products was subjected to a single cycle PCR reaction (denaturation at 95° C. for 10 min, annealing at 60° C. for 5 min and extension at 72° C. for 10 min) followed by a secondary PCR reaction (30 cycles; denaturation at 95° C. for 1 min, annealing at 60° C. for 1 min and extension at 72° C. for 1 min) using primers C5 and C4. The resultant PCR product of the Blo t 5-Der p 2 gene was gel purified and used for a second PCR with the pro-enzyme-Der p 1 PCR fragment and primers C1 and C4. The resultant PCR products for the three-in-one chimeric mite allergen D1proenzD1LB5LD2 with a size of 1.854 kbp were cloned into pGEMT- vector (Promega) to facilitate DNA sequencing using T7 and SP6 primers. The three-in-one chimeric mite allergen gene was subsequently cloned unidirectional into the Bam HI and Xba I site of a mammalian expression vector, pcDNA3.0 (Invitrogen).

C1/f
5′-CCCCCC GGA TCC CGG GCG AAC TGC GTG GTT TTA
AG-3′
C2/r
5′-GCT CCC GCC GCC GCT CCC GCC GCC CAG GAT CAC CAC
GTA CGG GTA-3′
C5/f
5′-GGG AGC GGC GGC GGG AGC GGC GGC CAG GAG CAC AAG
CCC AAG AAG-3′
C6/r
5′-GGA GCC CCC TCC GGA GCC CCC TCC CTG GGT CTG AAT
GTC CTT CAC-3′
C3/f
5′-GGC TCC GGA GGG GGC TCC GGA GGG GAT CAG GTG GAC
GTC AAG GAC-3′
C4/r
5′-CCC CCC TCT AGA TCA GTC GCG GAT CTT AGC GTG GGT
GGC-3′
Linker (GGGS GGGSGG)

Transfection and Selection of Stable CHO-K1 Cell Lines Producing the Three-in-One Chimeric Mite Allergen D1proenzD1LB5LD2

The CHO-K1 cells were grown in a 5.0% CO₂ incubator at 37° C., in culture flasks (NUNC) containing DMEM medium (Gibco) supplemented with 10% fetal bovine serum (FBS), 4 mM L-glutamine and Penicillin or Streptamycin. The codon optimized D1proenzD1LB5LD2 gene in pcDNA3.0 (0.8 μg) was transfected into CHO-K1 cells using LipofectAMINETm 2000 reagent (Invitrogen) according to the manufacturer's protocol. On the second day following transfection, the cells were selected with 700 μg/mL of Geneticin (G418, Sigma Aldrich) for a total of 19 days; the media were changed every 3 days. At days 13 and 19, the spent culture media was used for Western Immunoblot analysis. Stable and amplified chimeric clones were also obtained by limiting dilution in ten 96-well plates. A total of 166 CHO-K1 clones were obtained; these were expanded in 24 wells plate, and cells were harvested and stored in FBS with 10% DMSO (Sigma) in liquid nitrogen. Culture supernatants from these clones were used for screening on a Western Immunoblot assay.

SDS-PAGE and Western Immunoblot Analysis

The purified CHO-K1-expressed three-in-one chimeric mite allergen was separated on a 10% Tris-Tricine SDS-PAGE. After electrophoresis, proteins were electroblotted onto Hybond-C nitrocellulose membranes (Amersham Life Sciences, UK) using a semi-dry Transblot system (Pharmacia) at 100 mA for 1 h. The membrane was blocked in PBS-T (0.05% Tween-20) containing 3% skim milk for 1 h at room temperature. After an overnight incubation with mAb in blocking buffer at 4° C., the membrane was washed four times in PBS-T (5 min/wash) and incubated with biotinylated anti-mouse Ig (1:5,000 dilutions). The membrane was incubated with peroxidase conjugated ExtrAvidin (1:5,000 dilution) (Sigma, St Louis, Mo.) for 1 h at room temperature and subsequently developed in SuperSignal® West Pico Chemiluminescent Substrate (Pierce, USA) for 5 min.

Expression of Three-in-One Chimeric Mite Allergen in CHO-K1 Cells

Positive CHO-K1 clones were adapted to serum free EX-CELL™ 302 media (JRH Biosciences, Inc.) according to the manufacturer's protocol. A stable clone from serum supplemented media was first adapted to a decreasing FBS concentration in EX-CELL™ 302 media (5%, 1% FBS) by two successive passages each, and then in serum free EX-CELL™ 302 media supplemented with L-glutamine (4 mM) and Penicillin or Streptamycin. The adapted cells were subsequently grown in 200 mL suspension culture, seeded at 2×105 cells/mL in 500 mL shaker flasks, and shaken at 88 rpm in a 5.0% Co₂ incubator at 37° C. Spent culture media was harvested after 7 days of culture by centrifugation at 2000 rpm for 10 min and stored at −20° C. before purification.

Affinity Chromatography Purification of Three-in-One Chimeric Mite Allergen

Monoclonal antibody (mAb) against Der p 1 (4C1) (Indoor Biotechnologies, Inc., UK) was coupled to cyanogen bromide-activated Sepharose® 4B (Amersham Biosciences) according to the manufacturer's protocols. The resultant affinity column was used for purification of the three-in-one chimeric mite allergen from CHO-K1 spent culture medium, as previously described [20]. The column was first washed with 1× high-salt TBS (10 mM Tris, 0.5M NaCl) pH 7.5 until the OD₂₈₀ₙₘ became zero. The culture supernatant was added to the column and the flow-through was collected. Bound polypeptides were eluted in 5 mM Glycine 50% Ethylene Glycol (pH 10.0), collected in 0.5 ml fractions and neutralized in 0.1M sodium phosphate buffer (pH 7.0). Fractions containing the eluted three-in-one chimeric mite allergen were pooled and dialyzed in five changes of a total of 2.5 L of PBS (pH 7.4).

Absorption ELISA Assay

The three-in-one chimeric mite allergen was added to diluted samples of serum from a subject to a final concentration of 10 μg/mL and incubated at 4° C. for 16 h. The pre-absorbed sera were then reacted with plate-bound IgE at 4° C. for 16 h. The plate-bound IgE were developed as in the ELISA human IgE assay described previously [20]. The ELISA plates were coated overnight at 4° C. with 5 μg/mL of the respective yDer p 1, yDer p 2 or yBlo t 5 proteins in coating buffer. Plates were blocked with 1% BSA in PBS-T (1 h at room temperature) and incubated at 4° C. overnight with the pre-absorbed human sera in blocking buffer added in duplicate. Plates were then incubated with biotinylated anti-human IgE (Pharmingen CA) (1:2,000 dilutions) and subsequently developed as described above. The percentage of inhibition was calculated as (1−OD₄₀₅ₙₘ with inhibitor/OD₄₀₅ₙₘ without inhibitor)×100.

Skin Prick Test

Fifteen atopic subjects (including 3 children) with sera that are positive to one or two of the allergens or to all three allergens were recruited for this test. A drop of 10 pl of purified allergen (yDer p 1, yDer p 2, yBlo t 5) or crude extracts (Der p or Blo t) or the three-in-one chimeric mite allergen in normal saline (25 μg/mL) was applied on the volar side of the forearm followed by a skin prick with a disposable lancet. Histamine (1 mg/mL) and normal saline were included as positive and negative controls, respectively. The diameter of the wheal and/or erythema 30 min after the skin prick was measured and recorded. The appearance of a wheal and/or erythema with a diameter larger than the negative control by 3×3 mm was considered as a positive skin reaction.

Histamine Release Assay

CHO-K1 cell-expressed three-in-one chimeric mite allergen and a mixture of Der p 1; Der p 2 and Blot 5 allergens were diluted in a histamine release reaction buffer to concentrations ranging from 0.1 fM to 1 μM. Histamine release was performed with histamine release in heparinized whole blood kit (IBL, Hamburg, Germany). 200 μl heparinized whole-blood samples were incubated at 37° C. for 1 h with different concentrations of the allergens. The released histamine in the supernatant was subsequently determined using a specific plasma immunoassay, the histamine ELISA (IBL), according to the manufacturer's instructions.

Analysis of Cytokine Profiles

Blood was collected from both non-atopic and atopic subjects. Peripheral blood mononuclear cells (PBMCs) were obtained by the Ficoll-Paque centrifugation method. A total of 4×105 PBMC was cultured in AIM-V medium in the absence or presence of 1 μM Der p 1, Der p 2, Blo t 5, mixture of the three allergens, CHO-K1 cell-expressed three-in-one chimeric mite allergen or E. coli-expressed three-in-one chimeric mite allergens in a 96-well U bottom plate for 6 days, after which the supernatant was collected. The cytokine profile was analyzed using the Th1/Th2 cytokine kit from BD™ Cytometric Bead Array (BD Biosciences) according to the manufacturer's protocols.

Results and Discussion

Generation of Codon Optimized/Humanized Three-in-One Chimeric Mite Allergen vzv-D1proenzD1LB5LD2 Gene

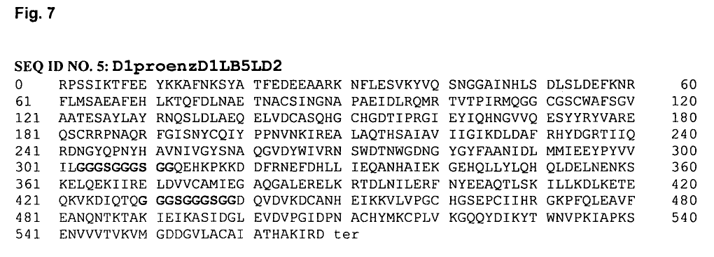

Optimization of codon usage has been shown to increase heterologous gene expressions in mammalian cells [21-24]. It has previously been shown that codon optimized Blo t 5 gene tagged to a VZV secretory signal in pCDNA3.0, yielded a high expression level in CHO-K1 cells [19]. A codon optimized chimeric gene consisting of three major allergens, Der p 1, Blo t 5 and Der p 2 linked by ten amino acid linker peptides was produced (FIG. 7). The nucleic acid sequence of the codons were replaced without changing the amino acid sequence in order to achieve a closer percentage frequency for individual codons to that of highly expressed human genes. This codon optimized chimeric gene fragment with a codon optimized VZV leader peptide at the N-terminal was generated by PCR using over-lapping oligonucleotide primers.

Transfection and Selection of Stable CHO-K1 Cell Lines Producing the Three-in-One Chimeric Mite Allergen D1proenzD1LB5LD2

A high-titer expression of recombinant polypeptide is dependent on the cell-line used, proper construction of the expression plasmid (promoter/regulatory sequences), the efficiency of transfection (electoporation or cationic lipids) and intergration at high transcription active sites within the genome by selection and amplification [21, 22]. A high level expression of codon optimized Blo t 5 chimeric gene in CHO-K1 cells using a conventional mammalian expression vector, pcDNA 3.0 was previously achieved by the inventors [19]. Expression was driven by a cytomegalovirus promoter and a Kozak sequence was also inserted before the AUG initiation codon to facilitate initiation of mRNA translation.

Selection of CHO-K1 transfectants with 700 ug/ml of G418 started a day after transfection. On day 5, most of the untransfected cells were dead and colonies became distinct on day 10. These colonies were allowed to expand for an additional 9 days in culture with three changes of fresh media (with G418). Western immunoblot analysis of spent culture media obtained on days 13 and 19 showed a positive band at approximately 66 kDa for chimeric gene transfectants. Subsequently, stable production cell lines of the three-in-one chimeric mite allergen were obtained by the cloning of homogeneous cell populations from heterogeneous cell pools by limiting dilution. A total of 161 supernatants of CHO-K1 cell clones were subjected to primary screening on a Western Immunoblot assay. 20 μl of each culture supernatant was separated on a 8% SDS-PAGE and transferred by Western blotting onto a Nitrocellulose membrane. Immunoblot analysis was performed using a mixture of monoclonal antibodies against Der p 1(1:5000), Blo t 5 (1:2000) and Der p 2 (1:10,000). Rat anti-mouse Ig biotin conjugate (1:5000) and Extravidin peroxidase (1:5000) diluted in 0.05% PBST were used as secondary and tertiary antibodies, respectively. Positive bands of the expected molecular weight (66.2 kDa) were detected in supernatant from 10 clones. In the secondary screening, positive clones were individually screened by probing with monoclonal antibodies against Der p 1, Blo t 5, or Der p 2 respectively, out of which five clones with different expression levels of the three-in-one chimeric mite allergen D1proenzD1LB5LD2 were obtained. Two clones, CHO-54 and CHO-80 were adapted to serum free EX-CEll™ 302 media and grown in 200 mL suspension culture for 7 days.

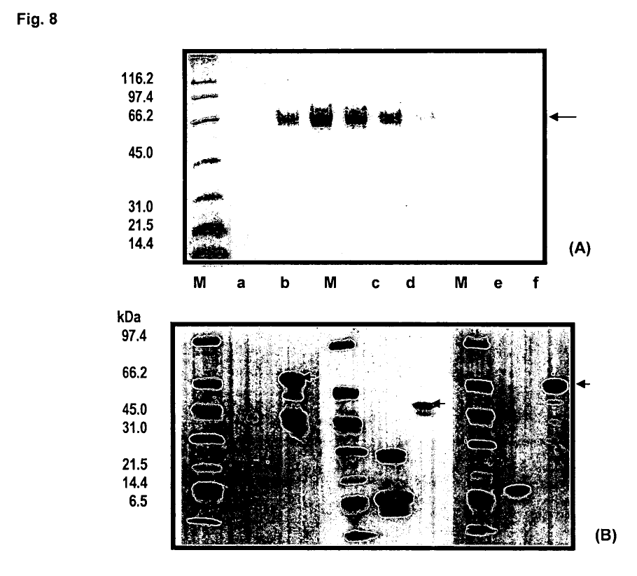

Both clones stably produced the three-in-one chimeric mite allergen in the absence of G418 indicating that selection of transfectant cells via G418 led to eventual intergration into the genome. The expressed three-in-one chimeric mite allergen was purified from spent culture media using monoclonal 4C1 affinity column, and showed a purity of more than 90% as determined on SDS-PAGE by Coomassie staining (FIG. 8). The protein was also analyzed on a Western Immunoblot probed with individual monoclonal antibodies against Der p 1, Der p 2 and Blo t 5 as described above (FIG. 8).

Skin Reactivity to Three-in-One Chimeric Mite Allergen

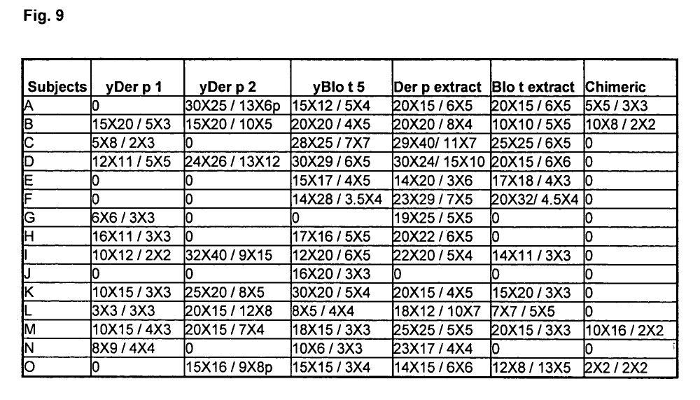

In-vivo allergenicity of CHO-K1-expressed three-in-one chimeric mite allergen D1proenzD1LB5LD2 was evaluated using skin prick tests on 15 atopic subjects with sera that are IgE positive for one or two of the allergens or to all three allergens. All subjects tested positive for histamine (1 mg/mL) and negative for the saline control. However, only four subjects (26.6%) showed positive skin reactivity to the three-in-one chimeric mite allergen while all showed decreased reactivity in both wheal and/or erythema size (FIG. 9).

Absorption ELISA Assay

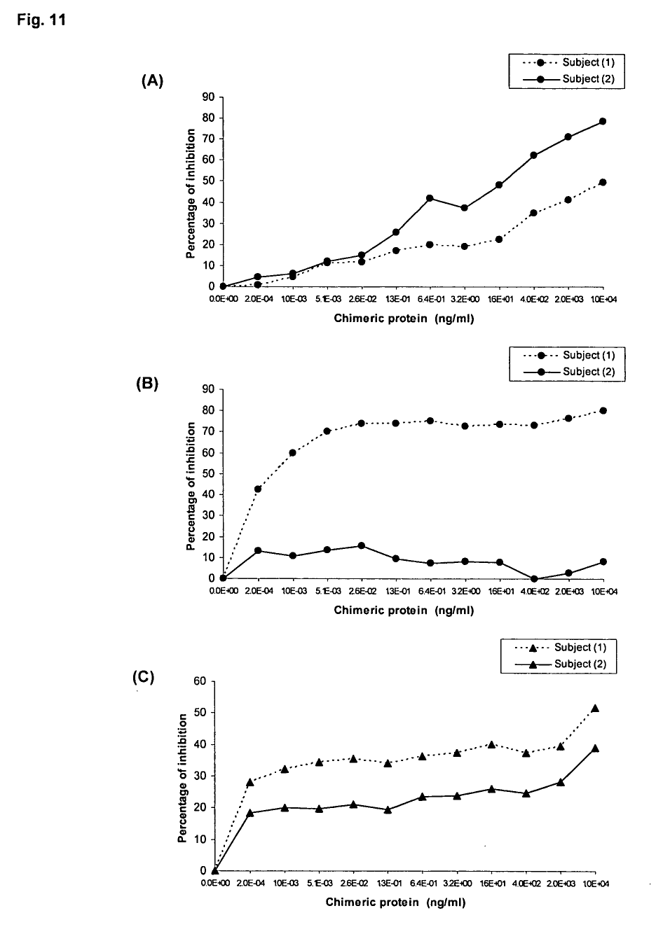

In the absorption study, sera from two atopic subjects were used; both having sera IgE to all three mite allergens, subjects 1 and 2 have high Der p 2-specific and Blo t 5-specific serum IgE titers, respectively. Sera were pre-absorbed with various concentrations of the three-in-one chimeric mite allergen before incubating with (A) yDer p 1, (B) yDer p 2 or (C) yBlo t 5 coated plates. As shown in FIG. 11, CHO-K1-expressed three-in-one chimeric mite allergen only showed 50% inhibition of Blo t 5-specific IgE for both subjects. In addition, the three-in-one chimeric mite allergen showed approximately 80% inhibition of Der p 2-specific IgE for subject 1 but no inhibition for subject 2. Similarly, the degrees of inhibition of Der p 1-specific IgE for subjects 1 and 2 were reduced (45% and 75%, respectively). The absorption study demonstrated that IgE recognition epitopes in the three mite allergens were disrupted when fused in the CHO-K1-expressed three-in-one chimeric mite allergen.

Cytokine Profile of Human PBMC

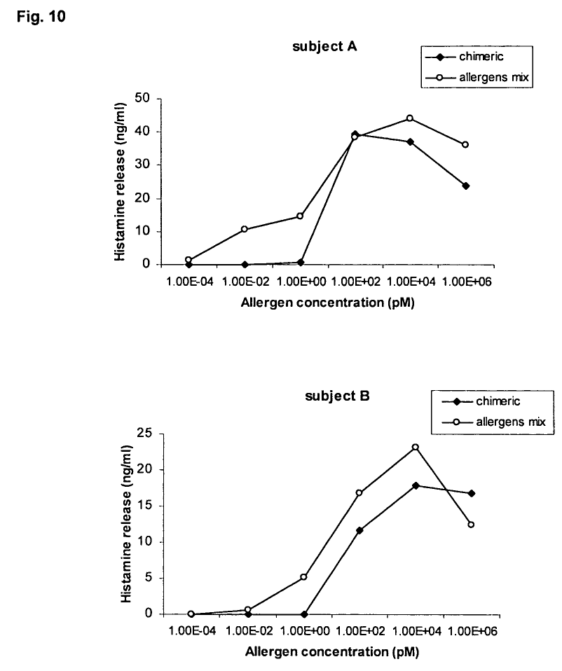

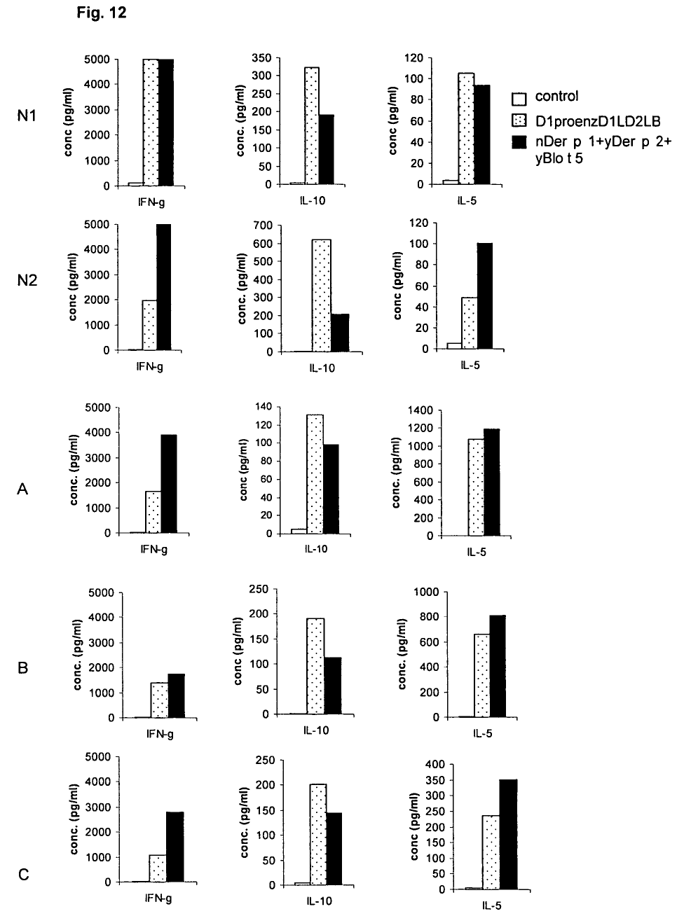

As shown in FIG. 12, the three-in-one chimeric mite allergen can induce other T-cell responses, i.e. production of similar levels of cytokine as that induced by a mixture of three individual allergens (Der p 1, Der p 2 and Blo t 5). The secretion of Th-2 cytokine (IL-5) and IL-10 induced by the three-in-one chimeric mite allergen or the mixture of three individual allergens is higher in atopic subjects (A, B, C) compared to normal subjects (N1 and N2). Similarly, E. coli-expressed GST-cleaved three-in-one chimeric mite allergen B5D2D1proD1 induced T-cell response with higher levels of IL-10 production (FIG. 5). In FIG. 10, CHO-K1-expressed three-in-one chimeric mite allergens required 104 folds of the allergen to induce histamine release in basophils from two different atopic individuals compared to the mixture of three individual allergens, indicating a decrease in allergenicity of the three-in-one chimeric mite allergens. Hence, both CHO-K1- or E. coli-expressed three-in-one chimeric mite allergens retain epitopes recognizable by human PBMCs and the ability to induce production of IL-10, an immuno-modulatory cytokine. Accordingly, both CHO-K1- or E. coli-expressed three-in-one chimeric mite allergens may be useful as immunotherapeutic reagents for dust mite associated allergic diseases. In addition, the CHO-K1-expressed three-in-one chimeric mite allergen showed a decrease in allergenicity compared to the mixture of three individual allergens, as indicated by the decrease in its ability to induce histamine release from basophilic leukocytes of two atopic individuals.

### APPLICATIONS

Five three-in-one chimeric mite allergens have been expressed in E. coli or CHO-K1 cells with high yield and purity. Clinical evaluation results based on human skin prick tests indicated that all the three-in-one chimeric mite allergens showed reduced or negative skin reactions, as compared with the unmodified individual mite allergens. In addition, data from histamine release assays showed that the ability of three-in-one chimeric mite allergen produced in CHO-K1 cells to induce IgE-mediated histamine release by human basophils was reduced by 100 times. Hence, these experimental data indicate that the three-in-one chimeric mite allergens are hypoallergenic (i.e. exhibit reduced ability to react to specific IgE), as compared with the unmodified individual mite allergens. The reduced IgE reactivity of these chimeric allergens may be used for developing safer immunotherapeutic reagents for allergy treatments.

Furthermore, all the three-in-one chimeric mite allergens retained their abilities to induce T cell responses as indicated by data from cell proliferation assay and cytokine ELISA. Advantageously, these three-in-one chimeric mite allergens are also able to increase production of IL-10. IL-10 is a T cell derived cytokine that down-regulates both Th1- and Th2-type responses and suppresses both IgE-mediated inflammation and delayed-type hypersensitivity inflammation. Together with IFN-gamma, it can also decrease the release of histamine and other mediators from mast cells and basophils. IL-10 is a potent suppressor of both total and allergen-specific IgE, and promotes B cell switching to IgG4 in the presence of IL-4. IL-10 has been regarded as the main cytokine in peripheral tolerance observed in venom, pollen and house dust mite immunotherapy. IL-10-producing cells have been detected in both the peripheral blood and in the nasal mucosa after immunotherapy.

The levels of IL-10 mRNA in allergen stimulated T cells of successfully treated patients with allergic rhinitis undergoing pollen SIT is higher than in those of poor or moderate outcome. The study also suggested that successful SIT depends on fast development and accessibility of IL-10 secreting T cells. A report of HDM-SIT in patients with house dust mite allergy demonstrated an increased in intracellular IL-10 production in CD4+ CD25+ T lymphocytes after 70 days of treatment [26, 27, and 28].

Advantageously, the three-in-one chimeric mite allergens of the invention may be used as effective and safe allergen-specific immunotherapeutic reagents for treatment of allergy by desensitization. In addition, as a protein-based booster, these reagents may also be incorporated into the DNA-prime-protein-boost approach for prophylactic and therapeutic DNA vaccines for mite allergy.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

### REFERENCES

1. Indoor allergens and asthma: report of the Third International Workshop. Platts-Mills T A, Vervloet D, Thomas W R, Aalberse R C, Chapman M D. J Allergy Clin Immunol. 1997 December; 100(6 Pt 1):S2-24. Review.
2. Pattern of Sensitization to Blomia tropicalis and its Recombinant Allergens in Four Tropical Asian Populations. Lim D L C, Shek L P C, Shaik W A, Baratawidjaja K, Trakultivakorn M, Pakit V, Cheong N, Chua K Y, Lee B W. Abstract. March 2002. A.A.A.I.
3. Importance of indoor allergens in the induction of allergy and elicitation of allergic disease. Custovic A, Simpson A, Woodcock A. Allergy 1998; 53(48 Suppl):115-20
4. Overview of allergy and allergic diseases: with a view to future. Br Med Bull. 2000; 56(4):844-64. Review.
5. Specificimmunotherapy. Larche M. Br Med Bull. 2000; 56(4):1019-36. Review.
6. Three years of specific immunotherapy with house-dust-mite extracts in patients with rhinitis and asthma: significant improvement of allergen-specific parameters and of nonspecific bronchial hyperreactivity. Pichler C E, Helbling A, Pichler W J. Allergy 2001 April; 56(4):301-6
7. Preventive aspects of immunotherapy: prevention for children at risk of developing asthma. Jacobsen L. Ann Allergy Asthma Immunol 2001 July; 87(1 Suppl 1):43-6
8. Specific immunotherapy in rhinitis and asthma. Bousquet J, Demoly P, Michel F B. Ann Allergy Asthma Immunol 2001 July; 87(1 Suppl 1):38-42.
9. Immunologic changes associated with allergen immunotherapy. Durham S R, Till S J. J Allergy Clin Immunol. 1998 August; 102(2):157-64. Review.
10. Skin test evaluation of genetically engineered hypoallergenic derivatives of the major birch pollen allergen, Bet v 1: results obtained with a mix of two recombinant Bet v 1 fragments and recombinant Bet v 1 trimer in a Swedish population before the birch pollen season. van Hage-Hamsten M, Kronqvist M, Zetterstrom O, Johansson E, Niederberger V, Vrtala S, Gronlund H, Gronneberg R, Valenta R. J Allergy Clin Immunol. 1999; 104(5):969-77.
11. Comparison of genetically engineered hypoallergenic rBet v 1 derivatives with rBet v 1 wild-type by skin prick and intradermal testing: results obtained in a French population. Pauli G, Purohit A, Oster J P, De Blay F, Vrtala S, Niederberger V, Kraft D, Valenta R. Clin Exp Allergy. 2000; 30(8):1076-84.
12. Engineering of the major house dust mite allergen Der f 2 for allergen-specific immunotherapy. Takai T, Yokota T, Yasue M, Nishiyama C, Yuuki T, Mori A, Okudaira H, Okumura Y. Nat Biotechnol. 1997 August; 15(8):754-8.
13. Hyposensitization to allergic reaction in rDer f 2-sensitized mice by the intranasal administration of a mutant of rDer f 2, C8/119S. Yasue M, Yokota T, Fukada M, Takai T, Suko M, Okudaira H, Okumura Y. Clin Exp Immunol. 1998 July; 113(1):1-9.
14. C8/119S mutation of major mite allergen Derf-2 leads to degenerate secondary structure and molecular polymerization and induces potent and exclusive Th1 cell differentiation. Korematsu S, Tanaka Y, Hosoi S, Koyanagi S, Yokota T, Mikami B, Minato N.J Immunol. 2000 September 1; 165(5):2895-902.
15. Immunoprophylaxis of allergen-induced immunoglobulin E synthesis and airway hyperresponsiveness in vivo by genetic immunization. Hsu C H, Chua K Y, Tao M H, Lai Y L, Wu H D, Huang S K, Hsieh K H. Nat Med. 1996 May; 2(5):540-4. Nat Med. 1996 May; 2(5):540-4.
16. Reversal of established CD4+ type 2 T helper-mediated allergic airway inflammation and eosinophilia by therapeutic treatment with DNA vaccines limits progression towards chronic inflammation and remodelling. Jarman E R, Lamb J R. Immunology. 2004 August; 112(4):631-42.
17. A major allergen gene-fusion protein for potential usage in allergen-specific immunotherapy. Kussebi F, Karamloo F, Rhyner C, Schmid-Grendelmeier P, Salagianni M, Mannhart C, Akdis M, Soldatova L, Markovic-Housley Z, Von Beust B R, Kundig T, Kemeny D M, Blaser K, Crameri R, Akdis C A. J Allergy Clin Immunol. 2005 February; 115(2):323-9.
18. Expression of Dermatophagoides pteronyssinus allergen, Der p II, in Escherichia coli and the binding studies with human IgE. Chua K Y, Dilworth R J, Thomas W R. Int Arch Allergy Appl Immunol. 1990; 91(2):124-9.
19. High-level expression of a codon optimized recombinant dust mite allergen, Blo t 5, in Chinese hamster ovary cells. L. H Lim., H. Y Li., N Cheong, B. W. Lee & K. Y. Chua (2004). Biochem. & Biophys. Res. Comm. 316: 991-996.
20. An extensive study of human IgE cross-reactivity of Blo t 5 and Der p 5 I. C. Kuo, N. Cheong, M. Trakultivakorn, B. W. Lee, K. Y. Chua, J. Allergy Clin. Immunol. 111 (2003) 603-609.
21. Expression of heterologous genes in mammalian cells, A. D. Levinson, Methods Enzymology, Academic Press, New York, 85 (1990) p 485.
22. Eukaryotic expression system: A comparison, S. Geisse, H. Gram, B. Kleuser, H. P. Kocher, Prot. Exp. and Purifi. 8 (1996) 271-282.
23. C. H. Kim, Y. Oh, T. H. Lee, Codon optimization for high-level expression of human erythropoietin (EPO) in mammalian cells, Gene 199 (1997) 293-301.
24. R. Bauer, M. Himly, A. Dedic, F. Ferreira, J. Thalhamer, A. Hartl, Optimization of codon usage increased immunogenicity of DNA vaccination of a major plant allergen in mice, Allergy 58 (2003) 1003-1010.
25. Schulz O, Sewell H F, Shakib F. Proteolytic cleavage of CD25, the alpha subunit of the human T cell interleukin 2 receptor, by Der p 1, a major mite allergen with cysteine protease activity. 187 J Exp Med. (1998) 271-275.
26. Jutel M, Akdis M, Budak F, Aebischer-Casaulta C, Wrzyszcz M, Blaser K, Akdis C A. IL-10 and TGF-beta cooperate in the regulatory T cell response to mucosal allergens in normal immunity and specific immunotherapy. Eur J Immunol. 2003; 33(5):1205-14.
Gardner L M, Thien F C, Douglass J A, Rolland J M, O'Hehir R E. Induction of T regulatory cells by standardized house dust mite immunotherapy: an increase in CD4+ CD25+ interleukin-10+ T cells expressing peripheral tissue trafficking markers. Clin Exp Allergy. 2004; 34(8):1209-19.
28. Nouri-Aria K T, Wachholz P A, Francis J N, Jacobson M R, Walker S M, Wilcock L K, Staple S Q, Aalberse R C, Till S J, Durham S R. Grass pollen immunotherapy induces mucosal and peripheral IL-10 responses and blocking IgG activity. J Immunol. 2004; 172(5):3252-9.

1. A chimeric polypeptide for reducing specific IgE binding to at least two house dust mite allergens in a subject, said chimeric polypeptide comprises an amino acid sequence comprising sequences of said at least two house dust mite allergens and wherein upon exposure to said allergens, said reduced binding causes reduced allergic reaction to said allergens in said subject.

2. The chimeric polypeptide according to claim 1, wherein said at least two house dust mite allergens are selected from the group consisting of Der p 1, Der p 2 and Blo t 5.

3. The chimeric polypeptide according to claim 2, wherein said Der p 1 comprises a Der p 1 prodomain.

4. The chimeric polypeptide according to claim 1 comprising the sequence set forth in SEQ ID NO. 1.

5. The chimeric polypeptide according to claim 1 comprising the sequence set forth in SEQ ID NO. 2.

6. The chimeric polypeptide according to claim 1 comprising the sequence set forth in SEQ ID NO. 3.

7. The chimeric polypeptide according to claim 1 comprising the sequence set forth in SEQ ID NO. 4.

8. The chimeric polypeptide according to claim 1 comprising the sequence set forth in SEQ ID NO. 5.

9. The chimeric polypeptide according to claim 1, wherein said subject is mammal.

10. The chimeric polypeptide according to claim 9, wherein said mammal is human.

11. The chimeric polypeptide according to claim 1, wherein said exposure is active exposure or passive exposure.

12. A polypeptide comprising a functional equivalent of a chimeric polypeptide according to claim 1, wherein said functional equivalent has at least 60% sequence identity with a polypeptide selected from the group consisting of SEQ ID NOs: 1, 2, 3, 4 and 5, and wherein said functional equivalent retains the reduced specific IgE-binding activity of the reference polypeptide.

13. The polypeptide according to claim 12, wherein the sequence identity is at least 70%, 80%, 90% or more.

14. A pharmaceutical composition comprising a chimeric polypeptide for reducing specific IgE binding to at least two house dust mite allergens in a subject, said chimeric polypeptide comprises an amino acid sequence comprising sequences of said at least two house dust mite allergens and wherein upon exposure to said allergens, said reduced binding causes reduced allergic reaction to said allergens in said subject.

15. The pharmaceutical composition according to claim 14, wherein said at least two house dust mite allergens are selected from the group consisting of Der p 1, Der p 2 and Blo t 5.

16. The pharmaceutical composition according to claim 14, wherein said chimeric polypeptide is selected from the group consisting of SEQ ID NOs. 1, 2, 3, 4, 5 and functional equivalents thereof.

17. The pharmaceutical composition according to claim 14, wherein said subject is mammal.

18. The pharmaceutical composition according to claim 17, wherein said mammal is human.

19. The pharmaceutical composition according to claim 14, wherein said exposure is active exposure or passive exposure.

20. A vaccine for reducing the severity of an allergic reaction to at least two house dust mite allergens in a subject, the vaccine comprising a chimeric polypeptide capable of reducing specific IgE binding to said at least two house dust mite allergens in said subject, said chimeric polypeptide comprises an amino acid sequence comprising sequences of said at least two house dust mite allergens and wherein upon exposure to said allergens, said reduced binding causes reduced allergic reaction to said allergens in said subject.

21. The vaccine according to claim 20, wherein said at least two house dust mite allergens are selected from the group consisting of Der p 1, Der p 2 and Blo t 5.

22. The vaccine according to claim 20, wherein said chimeric polypeptide is selected from the group consisting of SEQ ID NOs. 1, 2, 3, 4, 5 and functional equivalents thereof.

23. The vaccine according to claim 20, wherein said subject is mammal.

24. The vaccine according to claim 23, wherein said mammal is human.

25. The vaccine according to claim 20, wherein said exposure is active exposure or passive exposure.

26. A method for generating an immune response against at least two house dust mite allergens in a subject, the method comprising the step of administering to said subject a chimeric polypeptide according to claim 1.

27. A method of desensitizing a subject against house dust mite allergens, the method comprising the step of administering to said subject a chimeric polypeptide according to claim 1.

28. A method of reducing an allergic reaction to house dust mite allergens in a subject, the method comprising the step of administering to said subject a chimeric polypeptide according to claim 1.

29. Use of a chimeric polypeptide in the manufacture of a medicament for reducing specific IgE binding to at least two house dust mite allergens in a subject, said chimeric polypeptide comprises an amino acid sequence comprising sequences of said at least two house dust mite allergens and wherein upon exposure to said allergens, said reduced binding is capable of causing reduced allergic reaction to said allergens in said subject.

30. A method of producing the chimeric polypeptide of claim 1, the method comprising the steps of:
(a) providing a gene construct encoding said chimeric polypeptide in a suitable vector;
(b) transforming said vector into a suitable host cell;
(c) culturing said host cell under conditions which permit expression of said chimeric polypeptide; and
(d) collecting and purifying said chimeric polypeptide.

31. The method according to claim 30, wherein said vector is pGEX-4T or pcDNA3.0.

32. The method according to claim 30, wherein said host cell is E. coli or CHO-K1 cells.

33. A method for generating an immune response against at least two house dust mite allergens in a subject, the method comprising the step of administering to said subject a pharmaceutical composition according to claim 14.

34. A method for generating an immune response against at least two house dust mite allergens in a subject, the method comprising the step of administering to said subject a vaccine according to claim 20.

35. A method of desensitizing a subject against house dust mite allergens, the method comprising the step of administering to said subject a pharmaceutical composition according to claim 14.

36. A method of desensitizing a subject against house dust mite allergens, the method comprising the step of administering to said subject a vaccine according to claim 20.

37. A method of reducing an allergic reaction to house dust mite allergens in a subject, the method comprising the step of administering to said subject a a pharmaceutical composition according to claim 14.

38. A method of reducing an allergic reaction to house dust mite allergens in a subject, the method comprising the step of administering to said subject a vaccine according to claim 20.


SEQUENCE LISTING (13 sequences)

SEQ ID NO: 1 - protein, 470 aa, Escherichia coli
GSQEHKPKKDDFRNEFDHLLIEQANHAIEKGEHQLLYLQHQLDELNENKSKELQEKIIRELDVVCAMIEGAQGALERELKRTDLNILERFNYEEAQTLSKILLKDLKETEQKVKDIQTQDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDTNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 2 - protein, 550 aa, Escherichia coli
GSQEHKPKKDDFRNEFDHLLIEQANHAIEKGEHQLLYLQHQLDELNENKSKELQEKIIRELDVVCAMIEGAQGALERELKRTDLNILERFNYEEAQTLSKILLKDLKETEQKVKDIQTQDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDRPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 3 - protein, 550 aa, Escherichia coli
GSRPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVILQEHKPKKDDFRNEFDHLLIEQANHAIEKGEHQLLYLQHQLDELNENKSKELQEKIIRELDVVCAMIEGAQGALERELKRTDLNILERFNYEEAQTLSKILLKDLKETEQKVKDIQTQDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 4 - protein, 574 aa, Escherichia coli
GSRPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVILGGGGSGGGGSGGQEHKPKKDDFRNEFDHLLIEQANHAIEKGEHQLLYLQHQLDELNENKSKELQEKIIRELDVVCAMIEGAQGALERELKRTDLNILERFNYEEAQTLSKILLKDLKETEQKVKDIQTQGGGGSGGGGSGGDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 5 - protein, 568 aa, Cricetulus griseus
RPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVILGGGSGGGSGGQEHKPKKDDFRNEFDHLLIEQANHAIEKGEHQLLYLQHQLDELNENKSKELQEKIIRELDVVCAMIEGAQGALERELKRTDLNILERFNYEEAQTLSKILLKDLKETEQKVKDIQTQGGGSGGGSGGDQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 6 - DNA, 30 bp, Artificial Sequence
  synthetic construct
ggagggggctccggagggggctccggaggg

SEQ ID NO: 7 - DNA, 30 bp, Artificial Sequence
  synthetic construct
ggcggcgggagcggcggcgggagcggcggc

SEQ ID NO: 8 - DNA, 35 bp, Artificial Sequence
  primer
ccccccggatcccgggcgaactgcgtggttttaag

SEQ ID NO: 9 - DNA, 45 bp, Artificial Sequence
  primer
gctcccgccgccgctcccgccgcccaggatcaccacgtacgggta

SEQ ID NO: 10 - DNA, 45 bp, Artificial Sequence
  primer
gggagcggcggcgggagcggcggccaggagcacaagcccaagaag

SEQ ID NO: 11 - DNA, 45 bp, Artificial Sequence
  primer
ggagccccctccggagccccctccctgggtctgaatgtccttcac

SEQ ID NO: 12 - DNA, 45 bp, Artificial Sequence
  primer
ggctccggagggggctccggaggggatcaggtggacgtcaaggac

SEQ ID NO: 13 - DNA, 39 bp, Artificial Sequence
  primer
cccccctctagatcagtcgcggatcttagcgtgggtggc